(19) United States
(12) Patent Application Publication
(10) Pub. No.: US 2018/0133678 A1
(43) Pub. Date: May 17, 2018

(54) CAPSULES

(71) Applicant: LAMBSON LIMITED, Wetherby, Yorkshire (GB)

(72) Inventors: Andrew MINT, Farnborough, Hampshire (GB); Jonathan Richard VIRGOE, Guildford, Surrey (GB); David Vaughan PALMER, West Sussex (GB); Alexander Robin KERR, London (GB)

(21) Appl. No.: 15/574,671

(22) PCT Filed: May 10, 2016
(86) PCT No.: PCT/GB2016/051330

§ 371 Date: Nov. 16, 2017

(30) Foreign Application Priority Data
May 20, 2015 (GB) 1508658.0

(51) Int. Cl. B01J 13/10; C12P 13/14; C12P 19/26; C12P 39/00; C12N 1/16; A61K 9/50; A61K 47/44

(52) U.S. Cl. CPC B01J 13/10; C12P 13/14; C12P 19/26; C12P 39/00; A61K 47/44; A61K 9/5015; A61K 9/5036; A61K 9/5089; C12N 1/16

(57) ABSTRACT

A capsule having an encapsulated material and a capsule wall encapsulating the encapsulated material. The capsule wall includes a N-acetylglucosamine/glucosamine copolymer or a derivative of such a copolymer, wherein the N-acetylglucosamine/glucosamine copolymer or a derivative of such a copolymer is derived from a raw material which has a non-animal origin. The capsules have significant compression resistance while minimizing the amount of polymer incorporated into the capsule wall and are advantageously stable in a range of products including when associated with commercially available protease containing, biological liquid laundry products.

This invention relates to capsules and particularly, although not exclusively, relates to a process for producing capsules which comprise an encapsulated material.

Encapsulation is used to stabilize, isolate or protect materials from the external environment, enhancing the longevity of the encapsulated material, until such time as it is released. For example, oils sensitive to oxidation may be protected, or a volatile fragrance may be rendered inactive until released by some external force. Encapsulation allows formulations to be compounded with ingredients that would otherwise be unusable, due to incompatibility or reactivity.

Microencapsulation using complex coacervation has been known for some time, the basic concept being described in U.S. Pat. No. 2,712,507. Complex coacervation technology has been used commercially in large scale manufacture of carbonless copy paper and is still used today in this and wide range of other industries.

Multiple methods for performing encapsulation have since been developed and can be classified into physical and chemical methods. Physical methods consist of spray drying, spray chilling, fluid bed coating, extrusion and diffusion of materials into exhausted yeast or pollen particles. The chemical methods include simple and complex coacervation, interfacial polymerization, molecular inclusion and liposomes.

Complex coacervation has proved to be a versatile technology, providing the ability to produce both very small non-visual capsules (˜5 micrometres) up to large visual capsules in the region of 2 millimetres, with a wide array of capsule contents, such as vitamins, polyunsaturated fatty acids (PUFAs), essential oils, flavours, fragrances, cosmetic or health ingredients and pharmaceutical compounds.

The complex coacervation process is based on the electrostatic attraction of oppositely charged water soluble polymers, (simple coacervation refers to the use of only one polymer) which, in a controlled manner, can be made to form a wall or shell around very small emulsified droplets of hydrophobic materials such as oils. Finished capsules can be harvested and then applied to various substrates or dispersed into formulations where the core material can be stored until release is required.

Commonly used raw materials are gelatine (denatured collagen) and gum arabic (derived from the sap of Acacia trees). Gelatine is an amphoteric biopolymer, whose electrostatic charge is dependent on the pH of the environment. Anionic gum arabic (obtained from the sap of the acacia tree (Acacia Senegal)) is a commonly used counter-ion component, although alternative chemistries can be used, such as sodium carboxymethyl cellulose.

Both polymers are dissolved in water, in alkali conditions, at 50° C. The pH is lowered below the isoelectric point of the gelatine, with a weak acid, such as acetic acid, the charge on the gelatine becomes cationic and electrostatically interacts with the anionic polymer, forming the ‘coacervate’, a polymer rich, viscous phase. By carefully cooling the temperature of the system, the coacervate solubility decreases and, the viscosity of the coacervate increases and is deposited at the interface of the water and oil droplets, forming a continuous wall or shell.

The encapsulated oil droplets are then chemically stabilised with a crosslinking agent, such as formaldehyde, glutaraldehyde, or enzymatic treatments, thus isolating, protecting and stabilising the oils until the microcapsules are broken, releasing the contents.

The process of complex coacervation using gelatine and a counter ion is well known. In recent years coacervate capsules have been increasingly used in consumer goods, such as small, non-visual capsules used in anti-perspirant deodorants, fabric conditioners, hosiery and also large visual coloured microcapsules, in shower gels, toothpaste, handwash, etc.

However, gelatine/gum Arabic-based capsules have limitations in areas in which they may be used. For example, in situations where protease enzymes may come into contact with the capsules, such capsules cannot be used since the protease enzyme could prematurely break down the capsule.

In addition, the preparation of gelatin/gum Arabic capsules relies on dissolution of capsule wall materials at elevated temperature which can require substantial energy usage. Furthermore, the quality of a gelatine/gum Arabic-based system is only apparent after hours of controlled cooling. Consequently, if the quality of a batch is found to be poor, much time is expended producing an unusable batch. Additionally, it can be difficult to produce a narrow particle size distribution in the aforementioned gelating/gum-arabic-based system.

It is an object of the present invention to address the above-described problems.

It many situations, it is desirable to produce capsules with significant compression resistance whilst minimising the amount of polymer incorporated into the capsule wall. It is an object of the present invention to address this problem.

According to a first aspect of the invention, there is provided a capsule comprising an encapsulated material and a capsule wall encapsulating the encapsulated material, wherein the capsule wall includes a N-acetylglucosamine/glucosamine copolymer or a derivative of such a copolymer, wherein the N-acetylglucosamine/glucosamine copolymer or a derivative of such a copolymer is derived from a raw material which has a non-animal origin.

A material from which N-acetylglucosamine/glucosamine copolymer or a derivative of such a copolymer may suitably be a chitin. For the avoidance of doubt, a reference herein to a material which is derived (or cognate expressions) from chitin (or from any other material) does not exclude the possibility that a source material (e.g. chitin) may be subjected to one or more treatments to yield the material which is derived.

Chitin may be derived from a range of sources. For example chitin may be derived from crustaceans which in the context of the present specification is an animal source, Thus, for the avoidance of doubt, the reference to said “non-animal origin” excludes chitin derived from crustaceans. It suitably therefore excludes an N-acetylglucosamine/glucosamine copolymer or a derivative of such a copolymer which is derived from crustaceans.

Said chitin may be derived from a micro-organism. It may be derived from a fungus, for example, a yeast. It may be derived from a biomass. Said biomass may comprise zygomycetes, basidiomycetes, ascomycetes and deuteromycetes. In one preferred embodiment, said chitin may be derived from mycelium, for example of Aspergillus niger.

Said capsule wall preferably includes no animal-derived component. It preferably only includes components acceptable to vegetarians, especially acceptable to vegans.

Preferably, the entirety of said capsule includes no animal-derived component. It preferably only includes components acceptable to vegetarians, especially acceptable to vegans.

In said capsule, the ratio of the weight of encapsulated material divided by the weight of the capsule wall may be at least 3, preferably at least 5, more preferably at least 7, especially at least 8. The ratio may be less than 25 or less than 20.

Capsule and encapsulation processes described herein can be used to isolate, stabilise and protect sensitive ingredients or to control the release of active ingredients to a specific moment or point in a process, and may have utility in the following areas:
Personal care formulations, such as skin creams, emulsions, bodywashes, handwash, soaps, bodyscrubs, facial wipes, facial lotions, skin gels, shower gels, spritz formulations, lip treatments, antiperspirant deodorants, aftershave treatments, shaving preparations, fragrance delivery systems, including sprays, wipes, roll on, sticks. liquid & bar soaps, shower moisturisers, body/baby oil—other baby products including talc and nappy-rash creams.
Oral care preparations such as traditional toothpastes, toothpaste gels, mouthwash, dental floss, denture adhesive creams or pastes, denture treatment creams or tablets, plaque disclosure treatments, breath freshening strips, whitening strips, delivery of antimicrobials, whitening actives, flavours.
Cosmetic applications, such as, mascara, lipstick, eye shadow, nail varnish, other nail beneficiating treatments, synthetic nail applications, makeup removal treatments, including cotton wool pads and wipes. Foundation powders and sticks, tinted moisturisers, concealers, cream & powder blushers/highlighters, liquid & pencil eye-liners, temporary tattoos/decorations
Haircare preparations, such as, hair gels, hair mousses, styling aerosol sprays, anti-frizz treatments, hot oil treatments, pomades, powders, pastes, thermal damage repair treatments, hair removal creams or strips. Hair-care & styling split sprays & mousses into pump & aerosol preparations, shampoos, conditioners, dry shampoo, texturizing treatments, hair thickeners, hair growth treatments. Split end repair treatments.
Pharmaceutical preparations, such as transdermal patches, nasal sprays, mouthwashes, ointments, creams, gels, oral tablets and capsules, powdered formulations, eye drops, ear drops, suppositories, pessaries, smoking cessation aides, effervescent tablets, liquid delivery systems, such as gels, sachets, powders. Buccal delivery formulations. Impregnated bandages or pain relief formulation gels. Insect repellent formulations, sticks, liquids, sprays, wipes, wristbands and other impregnated fabrics.
Agricultural formulation, such as herbicides, pesticides, fungicides, nematodicides, rodenticides, insect repellents, insecticides, insect growth hormones, emulsifiable concentrates water dispersible granules, wettable powders or suspensions, seed treatments, such as water proofers and stickers, fertilisers plant food, nutrient delivery systems. Animal feed& supplements, vitamin delivery, teat disinfectants, veterinarian UV protection. Pet shampoo and conditioning. Medicated pet collars (e.g. fleas, ticks), veterinarian disinfectants. Medical and surgical gloves. Pet malodour treatments. Fruit surface treatments
Homecare products such as hard surface cleaners, furniture polish sprays, gels or wipes, floor cleaners (ready to use or concentrates), dust reduction spray or wipe formulations, plug in air fresheners, air freshener gels, air freshening candles, air or carpet freshening powders, air freshener sprays, antimicrobial wipes, dishwasher tablets or concentrated liquids, laundry detergent powders, sachets or liquids, fabric softener liquids of sachets, dye transfer inhibition liquids or sheets, antistatic or fabric beneficiating products, ironing water, ironing treatment sprays, hand dish liquids or gels, auto dish tablets or liquids, including rinse aides, bathroom cleaners, bleaches, limescale reduction formulations, stainless steel cleaners, mould or fungi reduction treatments, toilet cleaner concentrated liquids and gels, toilet block formulation, flush aides, biocides, kitchen/hob/oven cleaner/degreaser. Chrome polish, integrated floor cleaning/wipe systems. Malodour removal/reduction. Shoe and leather polishes and treatments. Waterproofing treatments for fabrics, textile and leather-treatments
Beverage formulations, such as carbonated soft drinks, still soft drinks, ready to drink fruit based drinks, squash concentrates, alcoholic beverages (spirits, wines, beers, ready to drink mixes), milkshake concentrates and ready to drink milkshakes (ambient and chilled), fruit juices, smoothies, hot beverages and infusions, such as tea, coffee, hot chocolate, flavoured waters (still or carbonated), mineral water (still or carbonated), energy drinks, dietary beverages (ready to drink or concentrates) powdered or gel versions of the above, sports recovery drinks,
Food preparations, such as baked pastry goods, cakes, breads, confectionary, chocolates, ice cream, desserts, ready-made meals, condiments, such as ketchup, mustard, salad dressings, soups, stews, broths. Emulsions, such as mayonnaise.
Dairy products, such as hard and soft cheeses, butter or spreads, yoghurts, crème fraiche, milk (skimmed, semi skimmed, unskimmed, UHT or pasteurised), buttermilk, whey protein based products, double or single cream, powdered whiteners for hot beverages. Non-dairy variants such as soy or almond milk.
Coating and adhesives, including solvent, water or powder based paints, both decorative and specialty (can coatings, automotive). Self-healing paint or coatings applications. Controlled release of biocides. Radiation cured coatings (automotive refinish systems, electronics, including nail varnish and artificial nail systems.) Adhesives systems, including water based or solvent based, both 1 pack or 2 pack systems. Paint strippers and diluents.
Metallurgy, metal quenching processing aides, acid pickling, electroplating, metal cleaners, degreasers other surface treatments.

Oil extraction, production and refining. Cementing process chemicals. Gas hydrate inhibition, shale swell prevention additives, delivery of oil field chemicals to the oil wells, fracking. Drill head lubricants, catalysts.
Plastics, processing aides for either thermosetting or thermoplastics, self-healing applications, fragrance or ‘active’ delivery.
Electronics, battery manufacture, radiation absorbing materials, printed circuit boards, visual display screens, fragrance delivery systems.
Industrial lubricants, membranes, emulsifiers or dispersants.
Printing and inks, lithographic printing, including font solutions, flexographic printing, ink jet printing, paper making process chemicals, such as sizing agents. Paper coatings used to improve printing performance post manufacture.
Construction, cements and concrete admixtures and post coatings, defoamers and processing aides, gypsum boards, thermal regulation applications (phase change materials), sealants, anti-corrosion applications.

In a preferred embodiment, said capsule wall includes:

(a) N-acetylglucosamine/glucosamine copolymer or a derivative of such a copolymer as described;

(b) a component (A) or a residue of component (A) after reaction or interaction with material referred to in (a); and, optionally,

(c) a cross-linking moiety which may be a residue of a component (B).

Component (A) may be as described in the second aspect. It is preferably a water-soluble polymer and/or a polysaccharide.

Component (B) may be as referred to in the second aspect.

In said preferred embodiment, the ratio of the wt % of components referred to in paragraphs (a) and (b) may be at least 3 and, preferably, is at least 5; and may be less than 10 or less than 8.

According to a second aspect of the invention, there is provided a method of making a capsule comprising an encapsulated material and a capsule wall encapsulating the encapsulated material, the method comprising:

(i) selecting a N-acetylglucosamine/glucosamine copolymer or a derivative of such a copolymer;

(ii) selecting a material to be encapsulated;

(iii) subjecting the N-acetylglucosamine/glucosamine copolymer or a derivative of such a copolymer and the material to be encapsulated to conditions which cause the N-acetylglucosamine/glucosamine copolymer or a derivative of such a copolymer to be incorporated into a capsule wall which surrounds the material to be encapsulated, thereby defining said capsule.

Said capsule may include any feature of the capsule of the first aspect.

Said N-acetylglucosamine/glucosamine copolymer or a derivative of such a copolymer is suitably derived from a raw material which is of non-animal origin as described in the first aspect.

Said N-acetylglucosamine/glucosamine copolymer or a derivative of such a copolymer is preferably derived from a chitin. Said chitin may be derived from a micro-organism. It may be derived from a fungus, for example a yeast. It may be derived from a biomass. Said biomass may comprise zygomycetes, basidiomycetes, ascomycetes and deuteromycetes. In one preferred embodiment, said chitin may be derived from mycelium, for example of Aspergillus niger.

Said N-acetylglucosamine/glucosamine copolymer or a derivative of such a copolymer is preferably prepared as described in WO03/068824, the content of which is hereby incorporated herein by reference.

The average molecular weight of said N-acetylglucosamine/glucosamine copolymer or a derivative of such a copolymer may be measured by Ubbelohde capillary visosimetry as described in WO03/068824. Said N-acetylglucosamine/glucosamine copolymer or a derivative of such a copolymer selected in step (i) may have an average molecular weight of at least 10 kDa, suitably at least 20 kDa, preferably at least 40 kDa, especially at least 60 kDa. The average molecular weight may be less than 300 kDa, suitably less than 150 kDa, especially less than 100 kDa.

Said N-acetylglucosamine/glucosamine copolymer or a derivative of such a copolymer may have a degree of acetylation of at least 0.1 mol %, suitably of at least 5 mol %, preferably at least 10 mol %. The degree of acetylation may be in the range 5 to 30 mol %. The degree of acetylation may be assessed by method KZ PT-CQ-101, of Kitozyme

Said N-acetylglucosamine/glucosamine copolymer or a derivative of such a copolymer may have a viscosity (1 wt % in acetic acid solution (mPa·s)), (measured by method KZ PT-CQ-102 of Kitozyme) of 1 to 40 mPa·s, for example 5-40 mPa·s or 5-25 mPa·s.

Said N-acetylglucosamine/glucosamine copolymer or a derivative of such a copolymer selected in step (i) suitably includes a moiety of structure

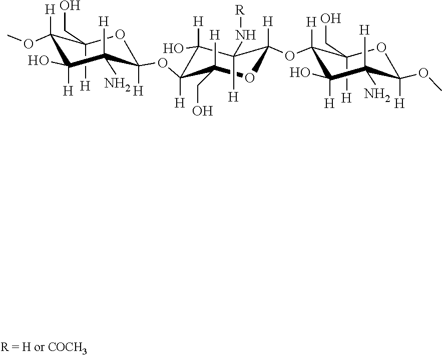

The method may include selecting a component (A) and subjecting the component (A) to conditions which cause it to be incorporated into said capsule wall. Said component (A) and said N-acetylglucosamine/glucosamine copolymer or a derivative of such a copolymer preferably define a coacervate phase which is arranged to encapsulate the material to be encapsulated in the method.

The method may include subjecting the N-acetylglucosamine/glucosamine copolymer or a derivative of such a copolymer and the component (A) to conditions such that they interact and/or react to define the capsule wall.

Said component (A) may be polar. Said component (A) may be anionic. Said component (A) may include carboxyl moieties. Said component (A) may be a polymer. Said component (A) may be an organic polymer. Said component (A) may be a cellulose or cellulose derivative, a polysaccharide, for example an anionic polysaccharide, a polyacrylate, an acrylic acid or methacrylic acid polymer, a polyphosphate, albumen or an albumen derivative, an alginate, a vinylacetate polymer, a vinylalcohol polymer, a gum for example carrageenan gum, xanthan gum or gum Arabic, an agar, a starch or a pectin.

Said component (A) is preferably a polymer. It is preferably a saccharide. It preferably includes a polysaccharide moiety. It is preferably a gum. It is preferably a naturally-occurring gum or a derivative thereof. It is preferably polar. It may be anionic. It preferably includes carboxyl moieties.

Said component (A) may be a hydrophilic polymer. Said component (A) is preferably water soluble.

Said material to be encapsulated is preferably hydrophobic. It is preferably more hydrophobic than the N-acetylglucosamine/glucosamine copolymer or a derivative of such a copolymer selected in step (i) of the method. It is preferably more hydrophobic than component (A) when provided.

Said material to be encapsulated may be an oil (e.g. mineral oil, silicone oil, a vegetable-derived oil, an essential oil, a fragrance oil), a butter (e.g. shea or coconut butter) or a wax (e.g. beeswax and carnauba wax).

The method of the second aspect preferably comprises providing said N-acetylglucosamine/glucosamine copolymer or a derivative of such a copolymer in an aqueous solution (A). Said component (A) is suitably also provided in solution in said aqueous solution (A). Thus, aqueous solution (A) suitably comprises said N-acetylglucosamine/glucosamine copolymer or a derivative of such a copolymer and said component (A). The ratio of the wt % of said component (A) divided by the wt % of said N-acetylglucosamine/glucosamine copolymer or a derivative of such a copolymer may be at least 3, preferably at least 5. It may be less than 10 or less than 8. The total wt % of dissolved solids in said solution (A) may be at least 5 wt %, preferably at least 7 wt %. It may be less than 20 wt %, less than 16 wt % or less than 12 wt %. Said solution (A) suitably has a pH as follows (or is adjusted to a pH as follows): greater than 1 and preferably less than 4 or less than 3. Aqueous solution (A) suitably is arranged to define a coacervate phase.

The method preferably comprising producing a formulation (B) which comprises aqueous solution (A) and said material to be encapsulated. The method may comprise contacting said material to be encapsulated with solution (A) to form formulation (B) and preferably stirring the combination. The method preferably therefore includes emulsifying or dispersing said material to be encapsulated in aqueous solution (A). Formulation (B) is suitably subjected to conditions whereby complex coacervation of the N-acetylglucosamine/glucosamine copolymer or a derivative of such a copolymer and component (A) occur. Such conditions suitably comprise increasing the pH of formulation (B). As the pH is increased, the N-acetylglucosamine/glucosamine copolymer or a derivative of such a copolymer may become less soluble and complex with component (A), thereby forming a coacervate phase which can coat the material to be encapsulated. Thus, in the method, a base is suitably contacted with formulation (B) to increase the pH, for example to a pH which is at least 0.5, for example at least 0.7 pH units higher than the starting pH. The pH may be greater than 2 or greater than 2.5 after contact with said base. It may be less than 4 or less than 3. At said pH, the capsule wall is suitably formed by the N-acetylglucosamine/glucosamine copolymer or a derivative of such a copolymer and said component (A).

Advantageously, it is found that formation of said capsule wall can be affected by the pH change described alone. In contrast, using other materials, a decrease in temperature is often required to induce wall formation and the integrity of the capsule wall cannot be assessed until after the wall has been cross-linked and/or cooled down to ambient temperature. Thus, in the method step of preferred embodiments of the present invention wherein the coacervate phase is formed, the temperature of formulation (B) does not increase to greater than 30° C. or greater than 40° C. or greater than 50° C.; and/or formulation (B) is not actively heated (i.e. formulation (B) is solely subjected to ambient conditions rather than any additional heat source).

After capsule wall formulation and/or after formation of said coacervate phase around said material to be encapsulated, the combination is suitably treated with a component (B) which is suitably arranged to increase the strength and/or integrity of the capsule wall and/or effect cross-linking (or other reaction) between components in the wall.

Component (B) may be arranged to react with hydroxy moieties in said capsule wall to effect cross-linking. Component (B) may be an aldehyde, for example having at least two aldehyde moieties. It may, for example, be glutaraldehyde. In general terms, component (B) may be selected from glutaraldehyde, formaldehyde, genepin, oleuropein, epichlorohydrin, tannic acid, gallic acid, sodium tripolyphosphate and transglutaminase.

Thereafter, the method preferably comprises recovering said capsule. Suitably, the method comprises making a multiplicity of capsules and recovering said multiplicity of capsules.

The method may comprise contacting said capsule or capsules with a preservative for preserving the capsules.

The method described is advantageous over prior methods in terms of the time taken to form capsules and its energy requirements. Furthermore, the capsules made have excellent physical and/or mechanical properties.

The invention extends, in a third aspect, to a capsule made in the method of the second aspect. The capsule may be as described in the first aspect.

According to a fourth aspect of the invention, there is provided a formulation comprising:

(i) capsule as described in any preceding aspect;

(ii) a protease enzyme or an alcohol.

Protease enzymes may be used in cleaning formulations to break down proteins. Alcohols may be included in a range of formulations, for example hand sanitisers. The capsules described herein may be advantageously used in formulations which contain protease enzymes or alcohols due to the relative stability of the capsule wall of the capsules in the presence of such enzymes or alcohols.

Specific embodiments of the invention will now be described by way of example, with reference to the accompanying drawings, in which:

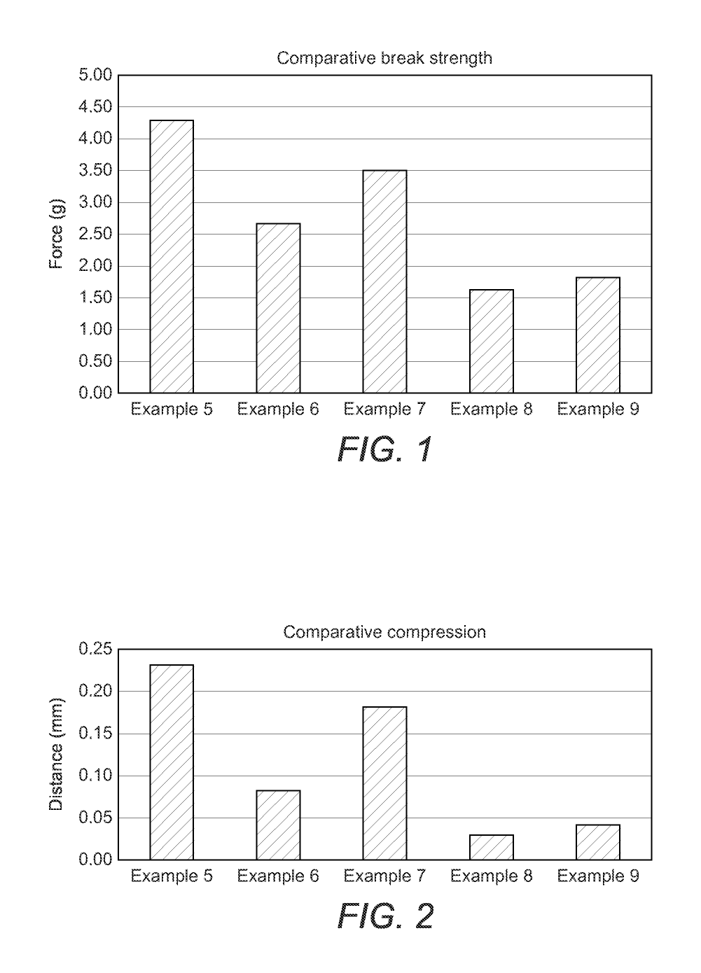

FIG. 1 is a graph comparing average break strengths of capsules made using different sources of chitosan;

FIG. 2 is a graph comparing compression before breakage of capsules made using different sources of chitosan;

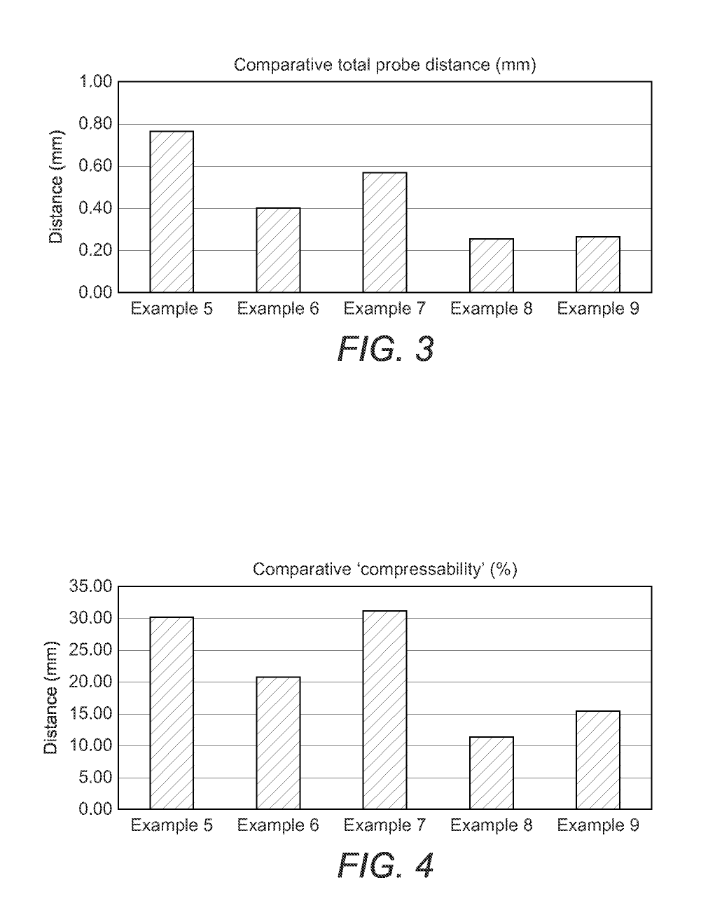

FIG. 3 is a graph comparing probe distance travelled before failure of capsules made using different sources of chitosan; and

FIG. 4 is a graph comparing compressibility of capsules made using different sources of chitosan;

The following materials are referred to hereinafter:

Copolymer (I)—biomass derived N-acetylglucosamine/glucosamine copolymer (commonly referred to as chitosan) having a molecular weight of 80 kDa obtained from Kitozyme.

Copolymer (II)—biomass derived N-acetylglucosamine/glucosamine copolymer having a molecular weight of 10-20 kDa obtained by from Kitozyme.

Marine Chitosan (HCMF)—obtained from Chitinor, Norway and having a molecular weight of 50-100 kDa.

Gum Arabic (Instant Gum) obtained from Nexrra, France.

Other reagents obtained from Sigma Aldrich.

Chitosan, or deacetylated chitin, is a linear copolymer comprised of randomly repeating glucosamine and N-acetylglucosamine units connected by β→(1,4) type linkages. The chemical structure is as shown below:

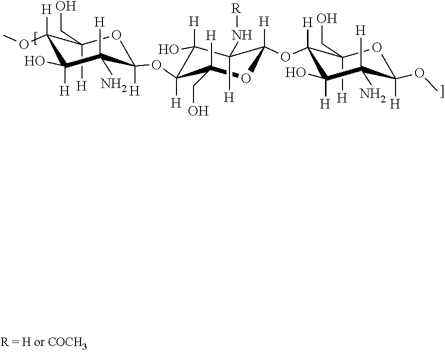

The N-acetylglucosamine/glucosamine copolymer is a positively charged polyelectrolyte and will undergo complex coacervation with negatively charged polyelectrolytes such as Gum Arabic. However, N-acetylglucosamine/glucosamine copolymer is not soluble above pH 5 (depending on degree of deacetylation) and therefore the controlled deposition of a coacervate cannot be achieved by adjustment of pH from neutral to acidic to allow precipitation to occur. Instead, a different approach can be utilised whereby coacervate is formed by controlling the charge on Gum Acabic Below pH 2.0, the ionisation of the carboxyl groups is minimal and an interaction with N-acetylglucosamine/glucosamine copolymer is not observed. By increasing the pH gradually, the anionic moieties on Gum Arabic become ionised which gives rise to interaction between N-acetylglucosamine/glucosamine copolymer and Gum Arabic and the coacervate can be seen as a concentrated liquid droplets, which can then be used to coat oil droplets. More particularly, a matrix encapsulation technique can be employed to create beads of N-acetylglucosamine/glucosamine copolymer encasing an active material. Material to be encapsulated is mixed into a solution of N-acetylglucosamine/glucosamine copolymer. Then, via a dropping mechanism, an alkali solution is dripped into the mixture which causes N-acetylglucosamine/glucosamine copolymer precipitation. The resultant beads which form entrap the active material in a spherical matrix, which can be recovered and maturated.

Microcapsules formed via complex coacervation will remain mechanically fragile or will maintain the potential to be re-solubilised by warm water or pH changes unless cross-linked. This crosslinking is achieved by application of a cross linking agent, which is typically a solution of glutaraldehyde or formaldehyde, although genepin, oleuropein, epichlorhydrin, sodium tripolyphosphate and transglutaminase have also been used.

In the following, Examples 1 and 2 describe the preparation of microcapsules using biomass derived N-acetylglucosamine/glucosamine (i.e. chitosan) and, subsequently, results of mechanical tests are provided.

### EXAMPLE 1—PREPARATION OF MICROCAPSULES MADE USING HIGH MOLECULAR WEIGHT COPOLYMER (I)

The internal oil phase component was:

High oleic sunflower oil (80 wt %)

β-carotene suspension (30 wt %) in vegetable oil (20 wt %).

The external water phase components comprised the following:

4 wt % solution (in deionised water) of Copolymer (I) (28.85 wt %).

16 wt % solution (in deionised water) of gum arabic solution (48.08 wt %).

Deionised water (23.07 wt %).

The external water phase components were combined (130 g) and mixed with an overhead stirrer (at 320 rpm), using a 4 blade star propeller in a beaker (400 ml), and adjusted to pH 1.7 with hydrochloric acid (25%, 2.3 g).

The internal oil phase components (100 ml) were mixed on a stirrer plate until homogenous. The internal oil phase was then added to the external water phase and the stirrer speed was increased to 1400 rpm for 120 seconds, yielding oil droplets with a modal average particle size of 50 microns. The stirrer speed was then reduced to 450 rpm.

In a separate beaker, 1 litre deionised water (480 ml) was adjusted to pH 1.7 with hydrochloric acid (25%). The emulsion comprising oil droplets was added to this beaker and stirring continued at 500 rpm.

Coacervation formation was as follows: A dropping funnel (50 ml) was charged with 50 mL of triethanolamine solution (5% wt in Deionised Water). The pH was monitored as the funnel was set to release approximately 0.5 ml/min. The emulsion was regularly checked under the microscope for wall formation and quality.

The flow of triethanolamine solution was stopped when the pH reached 2.78.

Glutaraldehyde solution (4 g, 50%) was then added to effect cross-linking. The solution was left to stir overnight.

The capsule suspension was transferred to another 2 litres beaker and diluted with deionised water. When the capsules had settled to the top, the suspension was filtered over 60 micron mesh fabric and washed with 4 litres of deionised water. The dry capsule slurry was then transferred to a beaker (400 ml) and weighed, yielding 125.68 g of approximately 85% solids. Preservative solution (87.97 g) was added, comprised of deionised water (98.5%), carboxy methylcellulose (1%) and potassium sorbate (0.5%), adjusted to pH 4.8 with citric acid solution (10%).

### EXAMPLE 2—PREPARATION OF MICROCAPSULES USING LOWER MOLECULAR WEIGHT COPOLYMER (II)

The internal oil phase component used was:

High oleic sunflower oil (80 wt %)

β-carotene suspension (30 wt %) in vegetable oil (20 wt %).

The external water phase component comprised the following:

4 wt % solution (in deionised water) of Copolymer II (28.85 wt %)

16 wt % solution (in deionised water) of gum arabic solution (48.08 wt %)

Deionised water (23.07 wt %)

The external water phase components were combined (130 g) and mixed with an overhead stirrer (at 320 rpm), using a 4 blade star propeller in a beaker (400 ml), and adjusted to pH 2.21 with hydrochloric acid (25%, 1.4 g).

The oil phase (100 ml) was then added to the external water phase and the stirrer speed was increased to 1200 rpm for 180 seconds, yielding an emulsion comprising oil droplets with a modal average particle size of 60 microns. The stirrer speed was then reduced to 400 rpm.

In a separate 1 litre beaker, deionised water (428 ml) was adjusted to pH 1.7 with hydrochloric acid (25%). The emulsion comprising oil droplets was added to this beaker and stirring continued at 500 rpm.

Coacervation formation was as follows:

A dropping funnel (50 ml) was charged with 50 mL of triethanolamine solution (5% wt in deionised water). The pH was monitored as the funnel was set to release approximately 0.33 ml/min. The emulsion was regularly checked under the microscope for wall formation and quality.

The flow of triethanolamine solution was stopped when the pH reached 3.17.

Glutaraldehyde solution (3 g, 50%) was then added to effect cross-linking. The solution was left to stir overnight.

The following test was used to assess the microcapsules.

### Test I—Microcapsule Mechanical Strength Determination

One capsule was isolated and centred underneath a probe (set at 5 mm height from plate) of a Stable Microsystem Texture Analyzer. The analyser was used to assess the degree of force required to break a single microcapsule.

After initiation of a test, the first drop in resistance to applied force was recorded. The test parameters (on Stable Micro Systems TA.XT plus Texture Analyser) were as follows:

Start height: 5 mm

Pre-test speed: 0.5 mm sec⁻¹

Test speed: 0.2 mm sec⁻¹

Trigger Force: 0.05 g

Maximum force: 100 g

Post-test speed: 10 mm sec⁻¹

Test sequence: return to start

Probe: Perspex cylinder

After a test, the probe and surrounding area were wiped down with a small amount of ethanol and a test repeated.

### Results

In tests, capsules of Example 1 were found to have significantly higher break strength compared to capsules of Example 2.

### EXAMPLES 3 AND 4—COMPARISON OF CAPSULES PREPARED USING BIOMASS DERIVED AND MARINE DERIVED CHITOSAN

Two batches of capsules based on a combination of Gum Arabic with different sources of chitosan were prepared and assessed.

The internal phase of the capsules consisted of the following:

| Constituent | Amount (wt %) |
|---|---|
| High oleic sunflower oil | 97.78 |
| Marula oil | 2 |
| Lutein suspension | 0.2 |
| Green 6 | 0.02 |

The Lutein and Green 6 colourants were included to make particle size determination easier.

The external phase of the capsules consisted of the following:

| Example | | Chitosan | Instagum | Deionised |
|---|---|---|---|---|
| No. | Chitosan Type | amount (g) | AA | water (g) |
| 3 | HCMF | 1.5 | 10 | 410 |
| 4 | Copolymer (I) | 1.5 | 10 | 410 |

For both Examples 3 and 4, the deionised water was lowered to pH 1.95 (21.5° C.) and the chitosan was added. When it had fully dissolved, the pH and viscosity were measured. The Gum Arabic was then added and stirred until dissolved, and hydrochloric acid (25% wt.) was used to lower the pH of the systems to 1.9, and the viscosity was again measured. Results are provided in the table below.

| | Example 3 | Example 4 |
|---|---|---|
| Time taken for full chitosan solvation | 9 minutes | 18 minutes |
| pH after chitosan solvation | 2.16 (22.2° C.) | 2.20 (21.9° C.) |
| Viscosity* (cP) after chitosan solvation | 32.4 (21.0° C.) | 22.6 (21.0° C.) |
| Time taken for full gum arabic solvation | 11 minutes | 7 minutes |
| pH after gum arabic solvation | 2.47 (22.5° C.) | 2.52 (22.1° C.) |
| Viscosity* (cP) after gum arabic | 30.4 (21.0° C.) | 22.6 (21.0° C.) |
| solvation | | |
| HCl (25% wt.) (g) to lower system to | 1.67 | 1.76 |
| starting pH | | |
| Starting pH | 1.88 (22.5° C.) | 1.87 (22.3° C.) |

*All measurement taken on Brookfield Viscometer, spindle 02, speed 100 rpm

The Example 3 formulation was clear, whereas the Example 4 formulation was a golden colour. The HCMF was faster to dissolve and had a higher viscosity than the Copolymer (I), though the Gum Arabic then took longer to dissolve in HCMF. For Example 4, approximately 5% more HCl (25% wt.) was required to achieve the appropriate pH.

The Internal Phases were then added, by being carefully dropped into the aqueous phase's stirring vortex, to prevent oil slick formation. The batches were stirred at 295 rpm.

Two dropping funnels were charged with triethanolamine (TEA) solution (5%), and set to drip around 1 ml/min. The TEA was then delivered. It was noted that the pH of the batches responded in lockstep with each other, proportionate to how much TEA solution had been added. The response of the encapsulations to TEA addition was noted and photomicrographs taken to record the progress.

After 20 ml of TEA addition, the two batches were around pH 2. No coacervate had yet formed in either batch. Local coacervate formation was macroscopically visible when TEA was added, at pH 2.18 for HCMF and Copolymer (I) at pH 2.28. However this did not translate to microscopically visible coacervate.

The first visible, lasting coacervate was for HCMF at pH 2.55, and Copolymer (I) at pH 2.56, though neither batch had yet formed walls. The Copolymer (I) batch had a finer, less visible coacervate at this point; the HCMF batch had more visible, larger coacervate.

At 78 ml of TEA addition, the pH was 2.63 for HCMF and 2.65 for Copolymer (I). Both batches had started forming walls around the smaller capsules. The Copolymer (I) coacervate was more discreet and the walls formed were clearer and more uniform.

Wall formation on all capsules began at pH 2.68 for HCMF, with thinner walls round the larger oil droplets. The coacervate appeared to drop in quality at this point, becoming less discrete droplets and more amorphous material from pH 2.68 up to the finishing pH of 2.85.

Walls formed more consistently around the various capsule sizes present in the Copolymer (I) batch. The coacervate remained recognisable as discrete droplets until a higher pH than HCMF, around pH 2.8.

At the final pH of 2.85, there were several differences between the batches. The Copolymer (I) batch was still producing coacervate locally upon addition of TEA solution. The walls were smoother and more consistent and optically clearer, and did not incorporate smaller oil droplets into the walls as the HCMF did.

HCMF stopped producing locally visible coacervate at pH 2.75. The walls appeared thicker than the Copolymer (I) capsules and less optically clear. The coacervate was more amorphous.

Both batches were then cross-linked with 6 g glutaraldehyde and stirred overnight. Both batches of capsules appeared stable and of good quality. The copolymer (I) batch had clearer walls and a lack of oil droplets incorporated into the walls; compared to the HCMF-based batch.

Yields were roughly the same: the filtered weight of the capsules was 121 g for HCMF and 119 g for Copolymer (I).

Each batch was split, with some half preserved in 0.5% xanthan, 0.5% rokonsal solution, and a quarter each in 0.5% rokonsal and either 2.5% CMC or 0.4% Gellan. The capsules were stable in Xanthan and Gellan.

The capsules were assessed and results obtained as described further below.

### Test (A)—Particle Size Comparison

Samples of xanthan preserved batches were taken and micrographs acquired. For each batch, five photographs and a total of 50 capsules were sized and the following results obtained.

| Capsules formed | Average | Particle |
|---|---|---|
| from components of | particle | size |
| Example No. | size (μm) | range (μm) |
| 3 | 555 | 206 to 866 |
| 4 | 440 | 139 to 787 |

### EXAMPLES 5 TO 9

The procedure described for Examples 3 and 4 was used to produce a range of particles for comparison as detailed in the table below.

| Example No. | Summary |
|---|---|
| 5 | Capsules formed from Copolymer I with modal average |
| | particle size of 1000 μm |
| 6 | Capsules made from Marine Chitosan HCMF with modal |
| | average particle sizes of 500 μm |
| 7 | Capsules formed from Copolymer I with modal average |
| | particle size of 500 μm |
| 8 | Capsules made from Marine Chitosan HCMF with modal |
| | average particle sizes of 250 μm |
| 9 | Capsules formed from Copolymer I with modal average |
| | particle size of 250 μm |

The capsules described in Examples 5 to 9 were further assessed for break strength, compression before breakage, total distance travelled by the probe used to apply the force to the capsules and compressibility (%). Results are presented in FIGS. 1 to 4. It is found that, in general, capsules based on Copolymer I have higher break strength and are generally more flexible or compressible compared to comparable capsules derived from marine chitosan.

As an alternative to the gum arabic, the following materials may be used:

Sodium carboxymethylcellulose or other cellulose derivatives, sodium polyacrylate, polyacrylic or methacrylic acid, sodium tripolyphosphate, albumen, alginates such as sodium alginate, alginic acid, polyphosphates, polyvinyl acetate, polyvinyl alcohol, carrageenan, casein, calcium caesinate, agar-agar, starch, pectins, Irish moss and xanthan gum.

The capsules prepared as described have been proven to offer reasonable stability (ie capsule walls remain intact and the capsules contents remain within the capsule) in commonly used personal care products, such as moisturising creams, shower gel, hand wash, shampoo and hydro-alcoholic formulations; in homecare applications, such as hand dish liquid, biological laundry detergent; in beverage formulations including dairy based beverages, like milkshakes and soft drinks, both still and carbonated.

Additionally, the capsules retain a relevant encapsulated material when associated with commercially available protease containing, biological liquid laundry products. Other prior art capsules were found to prematurely degrade, releasing the capsule content. Thus, the capsules can be advantageously used in protease enzyme-containing environments.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-32. (canceled)

33. A capsule comprising an encapsulated material and a capsule wall encapsulating the encapsulated material, wherein the capsule wall includes a N-acetylglucosamine/glucosamine copolymer or a derivative of such a copolymer, wherein the N-acetylglucosamine/glucosamine copolymer or a derivative of such a copolymer is derived from a raw material which has a non-animal origin.

34. The capsule according to claim 33, wherein the N-acetylglucosamine/glucosamine copolymer or a derivative of such a copolymer is derived from mycelium of a fungus; wherein in said capsule, the ratio of the weight of encapsulated material divided by the weight of the capsule wall is at least 8 and the ratio is less than 25; wherein said capsule wall includes:
(a) N-acetylglucosamine/glucosamine copolymer or a derivative of such a copolymer;
(b) a component (A) or a residue of component (A) after reaction or interaction with material referred to in (a) wherein component (A) is a water-soluble polymer; and
(c) a cross-linking moiety which cross-links components in the wall;
wherein the ratio of the wt % of components in (a) and (b) is at least 3 and is less than 10.

35. A method of making a capsule according to claim 33, the method comprising an encapsulated material and a capsule wall encapsulating the encapsulated material, the method comprising:
(i) selecting a N-acetylglucosamine/glucosamine copolymer or a derivative of such a copolymer, wherein said N-acetylglucosamine/glucosamine copolymer or a derivative of such a copolymer is derived from a micro-organism;
(ii) selecting a material to be encapsulated;
(iii) subjecting the N-acetylglucosamine/glucosamine copolymer or a derivative of such a copolymer and the material to be encapsulated to conditions thereby causing the N-acetylglucosamine/glucosamine copolymer or a derivative of such a copolymer to be incorporated into a capsule wall which surrounds the material to be encapsulated, thereby defining said capsule.

36. The method according to claim 35, wherein said N-acetylglucosamine/glucosamine copolymer or a derivative of such a copolymer selected in step (i) has an average molecular weight of at least 10 kDa and less than 300 kDa.

37. The method according to claim 35, wherein said N-acetylglucosamine/glucosamine copolymer or a derivative of such a copolymer has a degree of acetylation ranging from 5 to 30 mol %.

38. The method according to claim 35, wherein said N-acetylglucosamine/glucosamine copolymer or a derivative of such a copolymer has a viscosity of 1 to 40 mPa·s in 1 wt % in acetic acid solution.

39. The method according to claim 35, wherein a component (A) and said N-acetylglucosamine/glucosamine copolymer or a derivative of such a copolymer define a coacervate phase arranged to encapsulate the material to be encapsulated in the method, wherein said component (A) is polar and is a polymer.

40. The method according to claim 39, wherein said component (A) is a cellulose or cellulose derivative, or a gum.

41. The method according to claim 39, wherein said component (A) is a hydrophilic polymer; and said material to be encapsulated is hydrophobic.

42. The method according to claim 35, wherein said material to be encapsulated is an oil, a butter, or a wax.

43. The method according to claim 39, wherein the method comprises providing said N-acetylglucosamine/glucosamine copolymer or a derivative of such a copolymer in an aqueous solution (A); providing component (A) in solution in said aqueous solution (A); wherein the ratio of the wt % of said component (A) divided by the wt % of said N-acetylglucosamine/glucosamine copolymer or a derivative of such a copolymer is at least 3; and is less than 10; and wherein the total wt % of dissolved solids in said solution (A) is at least 5 wt %.

44. The method according to claim 43, wherein said solution (A) has a pH of greater than 1 or is adjusted to a pH of greater than 1; wherein the method comprises producing a formulation (B) which comprises aqueous solution (A) and said material to be encapsulated, wherein formulation (B) is subjected to conditions whereby complex coacervation of the N-acetylglucosamine/glucosamine copolymer or a derivative of such a copolymer and component (A) occurs, wherein said conditions comprise increasing the pH of formulation (B).

45. The method according to claim 44, wherein a base is contacted with formulation (B) to increase the pH, wherein the pH is greater than 2 and less than 4 after contact with said base.

46. The method according to any of claim 44, wherein when the coacervate phase is formed, the temperature of formulation (B) does not increase to greater than 30° C.; and/or formulation (B) is not actively heated.

47. The method according to claim 35, wherein after formation of a coacervate phase around said material to be encapsulated, the combination is treated with a component (B) which is arranged to effect cross-linking between components in the wall.

48. The method according to claim 47, wherein component (B) is arranged to react with hydroxy moieties in said capsule wall to effect cross-linking.

49. A formulation comprising:
(i) capsules according to claim 33; and
(ii) a protease enzyme or an alcohol.

50. The method according to claim 35, wherein said N-acetylglucosamine/glucosamine copolymer or a derivative of such a copolymer selected in step (i) includes a moiety of structure

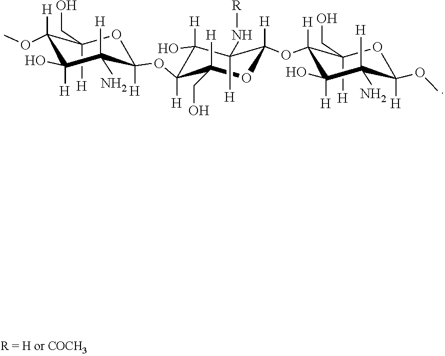

51. The method according to claim 35, wherein:
said N-acetylglucosamine/glucosamine copolymer or a derivative of such a copolymer selected in step (i) has an average molecular weight of at least 10 kDa and less than 300 kDa;
said N-acetylglucosamine/glucosamine copolymer or a derivative of such a copolymer has a degree of acetylation in the range 5 to 30 mol %;
a component (A) and said N-acetylglucosamine/glucosamine copolymer or a derivative of such a copolymer define a coacervate phase which is arranged to encapsulate the material to be encapsulated in the method, wherein said component (A) is a cellulose or cellulose derivative, or a gum;
said material to be encapsulated is an oil, a butter or a wax;
said N-acetylglucosamine/glucosamine copolymer or a derivative of such a copolymer selected in step (i) includes a moiety of structure

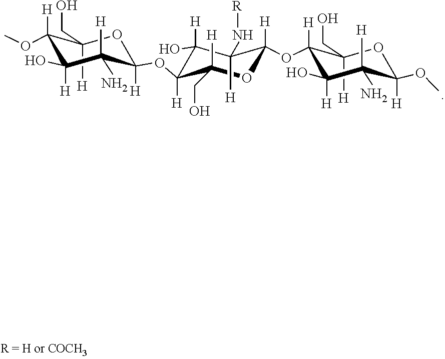

52. The method according to claim 35, wherein:
said N-acetylglucosamine/glucosamine copolymer or a derivative of such a copolymer selected in step (i) has an average molecular weight of at least 60 kDa and less than 150 kDa;
said N-acetylglucosamine/glucosamine copolymer or a derivative of such a copolymer has a degree of acetylation in the range 5 to 30 mol %;
said component (A) is a gum;
said material to be encapsulated is an oil, a butter or a wax;
the method comprises providing said N-acetylglucosamine/glucosamine copolymer or a derivative of such a copolymer in an aqueous solution (A); providing component (A) in solution in said aqueous solution (A); wherein the ratio of the wt % of said component (A) divided by the wt % of said N-acetylglucosamine/glucosamine copolymer or a derivative of such a copolymer is at least 3 and is less than 10; and wherein the total wt % of dissolved solids in said solution (A) is at least 5 wt %;
said solution (A) has a pH of greater than 1 or is adjusted to a pH of greater than 1; wherein the method comprises producing a formulation (B) which comprises aqueous solution (A) and said material to be encapsulated, wherein formulation (B) is subjected to conditions whereby complex coacervation of the N-acetylglucosamine/glucosamine copolymer or a derivative of such a copolymer and component (A) occurs, wherein said conditions comprise increasing the pH of formulation (B);
wherein a base is contacted with formulation (B) to increase the pH, wherein the pH is greater than 2 and less than 4 after contact with said base.
